(19) United States
(12) Patent Application Publication
(10) Pub. No.: US 2007/0179455 A1
(43) Pub. Date: Aug. 2, 2007

(54) Needle constructed with a transparent or translucent material

(76) Inventors: David Geliebter, Palm Beach Gardens, FL (US); Janelle R. Anderson, New York, NY (US)

Correspondence Address: CLARK & ELBING LLP, 101 FEDERAL STREET, BOSTON, MA, 02110, US

(21) Appl. No.: 11/638,837

(22) Filed: Dec. 14, 2006

Related U.S. Application Data
(60) Provisional application No. 60/751,379, filed on Dec. 16, 2005.
(60) Provisional application No. 60/762,321, filed on Jan. 26, 2006.

(51) Int. Cl. A61M 5/32

(52) U.S. Cl. 604/272

(57) ABSTRACT

In general, the invention features needles, with or without lumens, that contain a barrel being transparent or translucent or having a transparent or translucent portion. Needles of the invention may further include a lumen, the interior of which is visualizable through the transparent or translucent portion. A needle may also include a portion to which a suture is capable of being attached.

### CROSS-REFERENCE TO RELATED APPLICATIONS

This application claims benefit of U.S. Provisional Application Nos. 60/751,379, filed Dec. 16, 2005, and 60/762,321, filed Jan. 26, 2006, each of which is hereby incorporated by reference.

### BACKGROUND OF THE INVENTION

This invention relates to needles and methods of their use, e.g., for accessing the vasculature.

In known methods for either accessing a vessel or closing a wound, various needles are used. In the case of introducer needles, in order for the operator to ascertain that the needle has been properly placed in the vessel, as opposed to surrounding tissue or that the needle penetrated the vessel and then exited out the side of the vessel, it is common that the operator look for the presence of liquid, typically blood. In most cases this “visual flashback” takes time to appear out of the distal end of the needle, when the pressure levels are low, e.g., in introduction into a vein. In other cases, the pressure may be great enough, e.g., in an artery, so that this fluid is expelled out the distal end with enough force to expose the operator to this potentially hazardous fluid.

Moreover, to enter a vessel, the operator typically uses a small needle, e.g., 18 to 22 French, and when it is necessary to then enlarge the entry point to the vessel to accommodate a larger device, such a device would be introduced through a sheath placed over the needle. To achieve this end, it is often necessary to use a dilator, which is a tapered tubular device that fits over the needle, to dilate the size of the opening to a size sufficient to place the sheath or other device into the vessel without causing damage to the vessel.

With respect to solid surgical needles, these are usually made from a cut blank or a length of wire of a material such as stainless steel. The blank is then shaped using well known machining techniques to form the surgical needle and is therefore limited by virtue of the ability to properly shape the needle for its intended use, subject to the limitations of such metals and manufacturing techniques.

### SUMMARY OF THE INVENTION

In general, the invention features needles, with or without lumens, that contain a barrel being transparent or translucent or having a transparent or translucent portion.

Needles of the invention may further include a lumen, the interior of which is visualizable through the transparent or translucent portion. A needle may also include a portion to which a suture is capable of being attached. In various embodiments, the needle includes a scaffold adjacent to which the transparent or translucent portion is disposed. A needle may also include a metal tip, wherein the transparent or translucent portion is disposed distal to the tip. The metal tip may also be connected to a scaffold adjacent to which the transparent or translucent portion is disposed.

A needle of the invention may include an opaque tubular member having an opening therein, which is disposed adjacent the transparent or translucent portion. This needle may include first and second tubular members disposed coaxially, where the first tubular member includes the transparent or translucent portion, and the second tubular member includes the opaque tubular member. The first tubular member may be disposed within the lumen of the second tubular member, or the second tubular member may be disposed within the lumen of the first tubular member. A lumen of the needle may also be disposed within the first and second tubular members or between the first and second tubular members. Such a needle may also include a second lumen.

In another embodiment, the needle includes a first tapered region proximal to the tip, wherein the first tapered region is wider, i.e., has at least one larger cross-sectional dimension, than the tip. Such a needle may include two lumens not fluidically connected, wherein one lumen is fluidically coupled to the tip, and the other has an opening in the first tapered region. This needle may further include a second tapered region distal to the first tapered region and a third lumen that is not fluidically coupled to the other lumens and that has an opening in the second tapered region.

In various embodiments, a needle of the invention is beveled, e.g., having an A, B, or C bevel. A needle may also have a point style selected from the group consisting of Trephine, Cournand, Veress, Huber, Seldinger, Chiba, Trocar, Francine, Bias, Crawford, Deflected Tip, Hustead, lancet, and Tuohey. A needle may also be non-coring. In further embodiments, a needle, e.g., via the transparent or translucent portion thereof, is visualizable by ultrasound, fluoroscopy, x-ray, or other imaging techniques. In such embodiments, the transparent or translucent portion may include a radiopaque material or ultrasound responsive topography. A radiopaque material or ultrasound responsive topography that increases the contrast of a needle relative to the absence of the material or topography may also be included.

The invention also features a method for determining vascular access by inserting a needle of the invention having a lumen into a vascular vessel, e.g., an artery, of an animal, whereupon vascular fluid, e.g., blood, flows into the lumen and is visualized through the transparent or translucent portion. The needle may be resident in the animal from less than one hour to more than one day. The method may further include inserting a guidewire into the animal through the lumen. In another embodiment, the method includes dilating the opening of the vessel into which the needle is introduced and optionally inserting a catheter into the vessel through the dilated opening.

The invention also features a method of injecting or removing a fluid from an animal by inserting a needle of the invention having a lumen into the animal and introducing the fluid or removing the fluid from the animal. In various embodiments, the needle is inserted into a blood vessel, a body cavity, a muscle, a tendon, the skin, the spinal column, an eye, a wound, abscess, or an amniotic sac. The needle may include two lumens, and a fluid may be injected into the animal via one lumen, and a second fluid may be removed from the animal via the other lumen. The fluid introduced into the animal may include a drug, saline, or a vaccine, and the fluid removed from the animal may be blood, lymph, interstitial fluid, bile, amniotic fluid, cerebrospinal fluid, urine, pus, bone marrow, or ascites.

The invention also features a method for suturing by providing a needle of the invention having a portion to which a suture is capable of being attached and using the needle to suture an opening in an animal.

The invention further features a method of determining the presence or absence of a fluid in a needle of the invention including a lumen by monitoring the translucent or transparent portion for the presence or absence of the fluid.

By “proximal” is meant away from the tip of the needle. When the needle is introduced into a subject, the proximal end resides outside the body. Typically, the proximal end has a connector, e.g., an eye or a Luer lock.

By “distal” is meant towards the tip of the needle, e.g., the end through which liquid is taken up or expelled. When the needle is introduced into a subject, the distal end resides inside the body.

By “scaffold” is meant any supporting structure that has at least one lateral opening.

By “transparent” is meant transmitting light without appreciable scattering. The term includes materials that do not absorb light in the visible range and materials that absorb in the visible range so long as at least some visible light is transmitted.

By “translucent” is meant transmitting light with appreciable scattering. The term includes materials that do not absorb light in the visible range and materials that absorb in the visible range so long as at least some visible light is transmitted.

Other features and advantages will be apparent from the following description and the claims.

### BRIEF DESCRIPTION OF THE DRAWINGS

FIGS. 1-9 are schematic depictions of needles of the invention having lumens.

FIG. 10 is a schematic depiction of needles of the invention used for suturing.

The drawings are not necessarily to scale.

### DETAILED DESCRIPTION OF THE INVENTION

In general, the invention features needles having a transparent or translucent barrel or a barrel including a transparent or translucent portion. Reference to a transparent or translucent portion includes embodiments where the entire barrel is transparent or translucent, unless otherwise indicated. Furthermore, reference to a transparent portion excludes a void in the needle, although voids, such as eyes for threading sutures, may be additionally present. A needle of the invention may be in any form known in the art and may or may not have a lumen. In certain embodiments, a needle of the invention will have multiple lumens. Examples include a hypodermic needle or introducer needle. The tip of the needle may be beveled, pointed, blunt, or otherwise shaped. A needle may be single tipped or double tipped. For needles with lumens, one or more openings to the lumen may be disposed at the tip or on the side of the needle. The needle will be sized based on the material to be transferred and the substance into which the needle is inserted, if any, e.g., 11 to 32 gauge, e.g., 22, 21, 18, or 16 gauge, or 2 to 34 French. The needle may be of any length, e.g., ¼ to 6 inches. Furthermore, the needle may be attached to or otherwise have a connector, e.g., a Luer lock, slip tip, an eye, or other connectors known in the art.

A needle of the invention may have an A, B, or C bevel (GG-N-196), and it may have any point style known in the art. Exemplary point styles include Trephine, Cournand, Veress, Huber, Seldinger, Chiba, Trocar, Francine, Bias, Crawford, Deflected Tip, Hustead, and Tuohey. The point may also be a lancet. Furthermore, the needle may be non-coring, e.g., of tissue. The heel of the bevel may also be dulled either by sanding or by folding the metal over. A dulled bevel is advantageous in preventing skiving or other damage to a guidewire in the lumen.

The tip of a needle may also be visualizable by ultrasound, fluoroscopy, x-ray, or other imaging techniques. For ultrasound, the needle may include topographical patterns, e.g., lines of dimples, dents, and grooves, see, for example, U.S. Pat. No. 4,869,259. Such patterning may be present in the transparent or translucent portion or in any opaque portion of the needle. Desirably, the pattern is placed such that the tip of the needle is easily visualized in vivo. Needles may also have radiopaque portions, either in the translucent or transparent portion or in an opaque portion. Certain metals, e.g., used in the tip of a needle of the invention, are naturally radiopaque. Transparent or translucent materials may be radiopaque or rendered radiopaque by the inclusion of radiopaque additives to the portion, e.g., by doping a polymer or glass with Ba₂SO₄, tungsten, or any other suitable radiopaque material, by printing such materials on the transparent or translucent portion, or by otherwise fusing such materials to or embedding them in the translucent or transparent portion. Such additives or topographical patterning may also be employed to increase the visualizability of the needle, e.g., by increasing the contrast on the needle in vivo.

Materials used for the transparent or translucent portion of the needle may be any known in the art. Preferred materials include polymers such as polystyrene, polyethylene, polypropylene, polycarbonate, polyurethane, epoxy, polyamide (e.g., nylon), polyethylene terephthalic ester, polyvinyl chloride, polyacetate, polymethyl methacrylate, and styrene acrylonitrile copolymer. Additional materials include glass, silica, alumina, diamond, and sapphire.

In addition to the transparent or translucent portion, needles may contain other materials. Suitable materials include metals (e.g., stainless steel, titanium, chrome, shape memory alloy, e.g., nitinol, platinum, or nickel), ceramics, and opaque plastics.

Needles with lumens may be connected, either reversibly or irreversibly, to a vessel for holding fluid, e.g., a syringe or vaccutainer, a valve, or other component, e.g., tubing. Sutures and other threads may be connected to a needle of the invention by any means known in the art, e.g., through a standard eye or spring eye, where the eye is split to permit the eye to be forced over the suture, through an open channel into which the suture is swaged, through adhesives, or through an opening in the end of the needle into which the suture is inserted.

The transparent, translucent, or other portion of a needle may be formed by any suitable method. Exemplary methods include molding, photocuring, and machining. The use of plastics and other transparent or translucent materials allows for ease in forming a needle having desirable properties in any arbitrary shape. When a transparent or translucent material is employed in connection with another material, they may be attached by any method known in the art, including adhesives, screw threads, bolts, screws, friction fittings, and molding. In one embodiment, the transparent or translucent portion is molded around or otherwise attached to or supported by a scaffold, e.g., of metal. Exemplary shapes of scaffolds include a spiral, a mesh, e.g., random, square grid, or hexagonal, or other continuous, near-continuous, or non-continuous patterns, e.g., multiple vertical or horizontal strands/bars/threads, crisscross, wavy, or zig-zag pattern.

The following examples illustrate various embodiments of the invention.

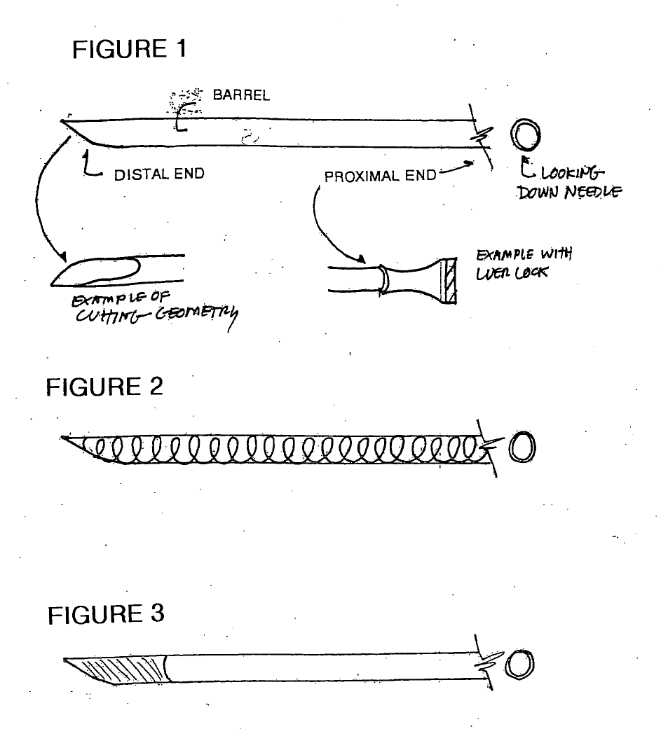

FIG. 1 shows a needle constructed of a transparent or translucent material. The tipped distal end that enters the body or vessel may have varied cutting geometry, as appropriate for the intended application. The design allows the user to see fluids, e.g., liquids, or any other materials in the needle, e.g., blood or medicine.

FIG. 2 shows a needle constructed from a transparent or translucent material that has an embedded or fused scaffold, i.e., the spiral structure depicted.

FIG. 3 shows a needle having a metal tip at the distal end and a transparent or translucent portion at the proximal end. The transparent or translucent portion may be overmolded to or permanently attached to the tip.

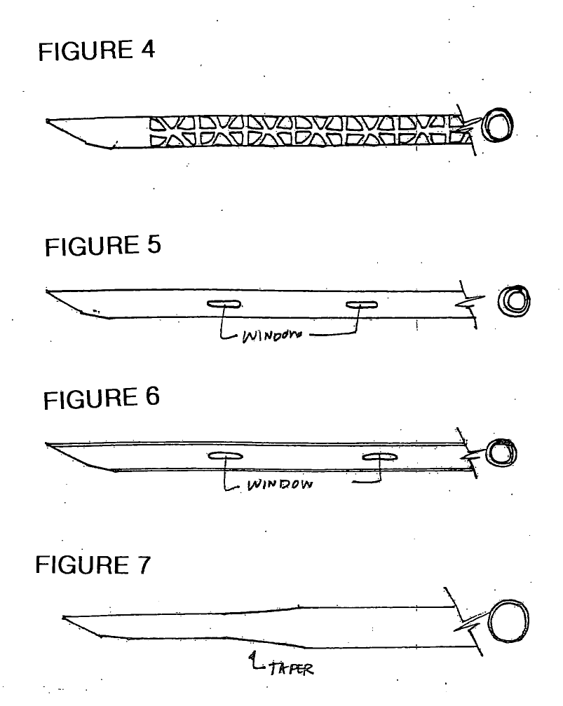

FIG. 4, similar to FIG. 3, shows a needle proximal end. A scaffold to which the transparent or translucent portion is overmolded or otherwise attached may also be connected to the metal tip, as depicted, or disposed proximal to the tip.

FIG. 5 shows a needle constructed of two tubular members, one within the other, with the inner member including a transparent or translucent portion, and the outer member being opaque (although it could also be transparent or translucent). The outer member has one or more openings, of any shape, which permit the user to visualize what is in the inner member, e.g., blood or medicine. These two members may be joined together to form one needle, although in various embodiments, they can act as either single or double lumen-needles.

FIG. 6 shows a needle similar to that of FIG. 5 in which the inner member is made of metal or other opaque material and the outer member has a transparent or translucent portion. The inner member has one or more openings that permit the user to visualize what is in the inner member, i.e., blood or medicine. These two tubes may be joined together to form one needle, although in various embodiments, they can act as either single or double lumen needles.

FIG. 7 shows a needle constructed of a transparent or translucent material, similar to that depicted in FIG. 1, wherein the needle includes a tapered region so the needle widens toward the proximal end. Such a needle may be used as a combination needle and dilator, where the user wishes to introduce a needle into a vessel or the body with a smaller size diameter needle and then enlarge the entry point, e.g., go from a 21 gauge to an 18 gauge. The exact taper shape and where the taper occurs may vary as per intended usage.

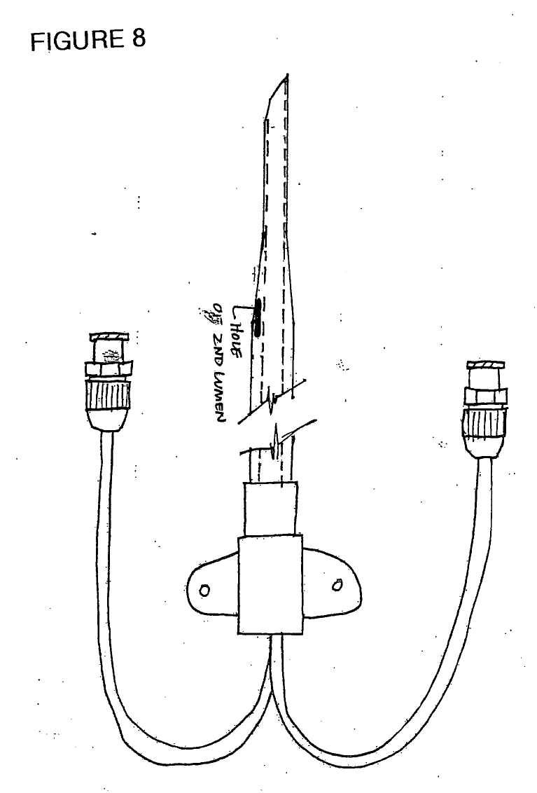

FIG. 8 shows a needle constructed of two or more parts. In its simplest embodiment, it has an inner needle that is similar to that depicted in FIG. 1, i.e., constructed of a transparent or translucent material and of constant diameter. Over this inner needle is one or more fitted tubes that are tapered at the distal end to fit tightly around the inner tube or needle and allow for the user to dilate the size of the entry hole, similar to FIG. 7. The fitted tubes around the inner needle may also contain a second lumen to allow simultaneous introduction, removal, or a combination thereof, or fluids or other materials from the multiple lumens in the needle.

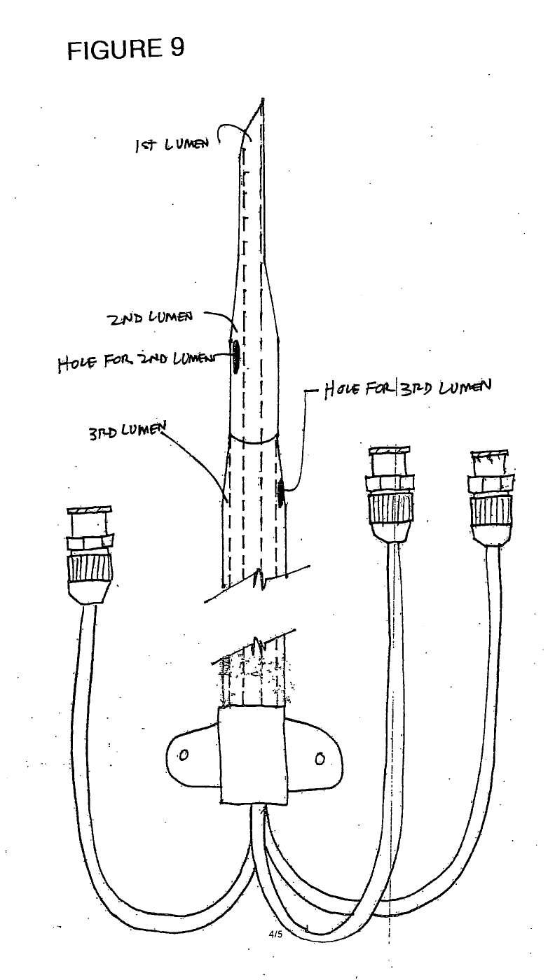

Similar to FIG. 8, FIG. 9 shows a needle having an inner needle and two fitted tubes to produce a needle having three lumens. Further fitted tubes may be added, e.g., to produce a needle having five lumens.

Although the above examples focus on needles having a lumen, similar designs are possible for needles not having a lumen.

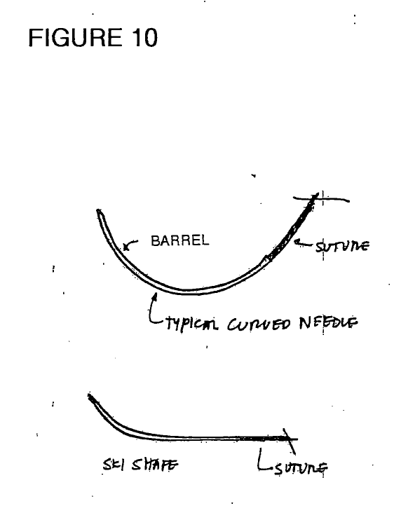

FIG. 10 shows a medical needle (e.g., surgical or cardiovascular) including a transparent or translucent material. Such a needle may be hollow although it may not have an opening to access the hollow portion. The needle may also include a metal tip or scaffold as with the needles having lumens described in FIGS. 1-9. The shape of the needle may vary, depending on its intended use, e.g., straight, half-curve (or ski), where the body is generally straight but curved near the point, or curved, formed by an arc of ¼, ⅜, ½, or ⅝ of a circle.

Methods

The needles of the invention may be employed in any suitable manner. Typically, needles with lumens will be used to transfer fluids, e.g., for injection or removal. Such a needle may be inserted into an animal, e.g., a mammal such as a human. Once inserted into an animal, a fluid (e.g., containing a drug, nutrients (e.g., in total parenteral nutrition), saline, blood or a blood product (e.g., plasma), or vaccine) may be injected into the animal or a fluid (e.g., a bodily fluid, such as blood, tears, saliva, lymph, interstitial fluid, bile, amniotic fluid, cerebrospinal fluid, mucous, urine, semen, pus, marrow, or ascites) may be removed from the animal. Cells or cellular samples may also be transferred via a needle of the invention. For example, needles of the invention may be employed to transfer cells in culture or to obtain needle biopsies. Gases, e.g., air, expired air, nitrogen, oxygen, and nitrous oxide, may also be injected or removed via needles of the invention. Needles may also be employed to allow access of a catheter, guidewire, endoscope, or other medical device, either through a lumen, over the needle, or after removal of the needle. Needles may be inserted into any appropriate portion of the animal including blood vessels, muscles, tendons, bones, skin, eye, other organs, body cavities, spinal column, wounds, abscesses, and amniotic sacs. The needle may be placed in the animal transiently, e.g., for less than 1 hour, or for an extended period of time, e.g., more than one day, such as with a heplock.

When used to transport fluids, the transparent or translucent portion of the needle desirably allows for visualization of the interior of the needle. The color of the fluid in the needle may be the basis of visualization. For example, arterial (or oxygenated) blood and venous (or deoxygenated) blood have characteristic colors, which may be indicative of their presence in a needle. The transparent or translucent portion may also be tinted such that the combination of the color of the fluid in the needle and the tinted portion is visualized. For example, a green tinted portion may be employed for use in needles carrying arterial blood. In other embodiments, the presence of a fluid in a needle may be visualized by a change in index of refraction, reflection, scattering, absorbance, fluorescence, luminescence, or phosphorescence. The visualizable property may be intrinsic to the fluid, or it may be the result of a chemical reaction between the fluid and a reagent, e.g., that is resident in the lumen. For example, luminol may be present in the lumen, either coated on the surface or in solution, and may react with blood entering the lumen to produce luminescence. The visualizable property of the fluid may be that of the fluid whose entry into or exit from the lumen is being monitored or that of a visualized fluid present in the lumen that is displaced by the entry into or exit from the lumen of the fluid being monitored. In one example, a liquid is placed in the lumen of the needle, and its displacement by a gas is visualized. The visualizable property of the fluid in the needle may be monitored by eye or by a photodetector (e.g., photodiode, photomultiplier tube, photographic film, photovoltaic cell, or CCD detector).

Visualization allows for confirmation that fluid is flowing, present, or absent in the lumen. Confirmation is especially useful when fluids are being removed from an animal. For example, when a needle is employed to gain vascular access, it is desirable to know as soon as possible when a vein or artery is punctured. By employing a needle of the invention, this visualization, or flash, occurs quickly, thereby reducing or eliminating loss of blood or exposure of an operator to potentially hazardous fluids. The visualization is also useful in determining whether fluid is flowing through the needle in general, e.g., to gauge when an entire dose of a drug, saline, or vaccine is administered or to gauge when all fluid has been removed from an area, e.g., a wound or abscess.

In one example, the needle is employed to gain access to blood vessels for introduction of a medical device, e.g., a cardiac catheter for angioplasty. The needle of the invention is introduced into a blood vessel, e.g., an artery. Typically, a dilator is then passed over the needle to dilate the opening. Alternatively, the needle includes a dilator, as described herein, which is advanced into the vessel. A sheath may then be advanced over the needle and dilator. A guidewire or other device may also be introduced via the lumen of the needle. The needle may then be removed, or it may remain in the patient. In either case, a medical device, such as a catheter, is inserted into the vascular access point initially created by the needle.

Suitable needles of the invention may also be used to pierce any suitable material, e.g., to introduce openings into a material, for acupuncture, or to suture (or sew) a material, e.g., a wound. As described above, the suture may be attached to the needle by any means. When non-permanent attachments are employed, the needles may be reused, including any necessary sterilization procedures. The needles may also be disposable, especially when constructed of inexpensive polymers.

### OTHER EMBODIMENTS

Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A needle comprising a barrel comprising a transparent or translucent portion.

2. The needle of claim 1, wherein said needle further comprises a first lumen and said transparent or translucent portion allows visualization of the interior of said first lumen.

3. The needle of claim 2, further comprising a scaffold adjacent to which said transparent or translucent portion is disposed.

4. The needle of claim 2, further comprising a metal tip, wherein said transparent or translucent portion is disposed proximal to said tip.

5. The needle of claim 4, wherein said metal tip is connected to a scaffold adjacent to which said transparent or translucent portion is disposed.

6. The needle of claim 2, further comprising an opaque tubular member having an opening therein, wherein said opening is disposed adjacent said transparent or translucent portion.

7. The needle of claim 6, wherein said needle comprises first and second tubular members disposed coaxially, said first tubular member comprises said transparent or translucent portion, and said second tubular member comprises said opaque tubular member.

8. The needle of claim 7, wherein said first tubular member is disposed within the lumen of said second tubular member.

9. The needle of claim 7, wherein said second tubular member is disposed within the lumen of said first tubular member.

10. The needle of claim 7, wherein said first lumen is disposed within said first and second tubular members.

11. The needle of claim 7, wherein said first lumen is disposed between said first and second tubular members.

12. The needle of claim 7, further comprising a second lumen.

13. The needle of claim 2, further comprising a first tapered region proximal to the tip, wherein said first tapered region is wider than said tip.

14. The needle of claim 13, further comprising a second lumen that is not fluidically coupled to said first lumen, wherein said first lumen is fluidically coupled to the tip, and said second lumen has an opening in said first tapered region.

15. The needle of claim 14, further comprising a second tapered region distal to said first tapered region and a third lumen that is not fluidically coupled to said first or second lumens and that has an opening in said second tapered region.

16. The needle of claim 2, wherein said needle is beveled.

17. The needle of claim 16, wherein said needle has an A, B, or C bevel.

18. The needle of claim 2, wherein said needle has a point style selected from the group consisting of Trephine, Cournand, Veress, Huber, Seldinger, Chiba, Trocar, Francine, Bias, Crawford, Deflected Tip, Hustead, lancet, and Tuohey.

19. The needle of claim 2, wherein said needle is non-coring.

20. The needle of claim 2, wherein said needle is visualizable by ultrasound, fluoroscopy, or x-ray.

21. The needle of claim 20, said transparent or translucent portion is visualizable by ultrasound, fluoroscopy, or x-ray.

22. The needle of claim 20, wherein said transparent or translucent portion comprises a radiopaque material or ultrasound responsive topography.

23. The needle of claim 20, further comprising a radiopaque material or ultrasound responsive topography that increases the contrast of said needle relative to the absence of said material or topography.

24. The needle of claim 1, further comprising a portion to which a suture is capable of being attached.

25. A method for determining vascular access comprising inserting the needle of claim 2 into a vascular vessel of an animal, whereupon vascular fluid flows into said first lumen and is visualized through said transparent or translucent portion.

26. The method of claim 25, wherein said needle is inserted into an artery and said vascular fluid is blood.

27. The method of claim 25, wherein said needle is resident in said animal for less than one hour.

28. The method of claim 25, wherein said needle is resident in said animal for more than one day.

29. The method of claim 25, further comprising inserting a guidewire into said animal through said first lumen.

30. The method of claim 25, further comprising dilating the opening in said vessel into which said needle is introduced.

31. The method of claim 30, further comprising inserting a catheter into said vessel through said dilated opening.

32. A method of injecting or removing a fluid from an animal comprising inserting the needle of claim 2 into said animal and introducing said fluid or removing said fluid from said animal.

33. The method of claim 32, wherein said needle is inserted into a blood vessel, a body cavity, a muscle, a tendon, the skin, the spinal column, an eye, a wound, abscess, or an amniotic sac.

34. The method of claim 32, wherein said needle further comprises a second lumen.

35. The method of claim 34, wherein said fluid is injected into said animal via said first or second lumen, and a second fluid is removed from said animal via the other lumen.

36. The method of claim 32, wherein said fluid is introduced into said animal.

37. The method of claim 36, wherein said fluid comprises a drug, saline, or a vaccine.

38. The method of claim 32, wherein said fluid is removed from said animal.

39. The method of claim 38, wherein said fluid is blood, lymph, interstitial fluid, bile, amniotic fluid, cerebrospinal fluid, urine, pus, bone marrow, or ascites.

40. A method for suturing comprising the steps of providing a needle of claim 16 and using said needle to suture an opening in an animal.

41. A method of determining the presence or absence of a fluid in a needle of claim 2 comprising monitoring said translucent or transparent portion for the presence or absence of said fluid.
